(19) United States
(12) Patent Application Publication
(10) Pub. No.: US 2023/0103548 A1
(43) Pub. Date: Apr. 6, 2023

(54) MECHANOACTIVE MATERIALS AND USES THEREOF

(71) Applicant: KNOTHE TATE, Melissa, New South Wales (AU)

(72) Inventor: Melissa Knothe Tate, New South Wales (AU)

(21) Appl. No.: 17/909,675

(22) PCT Filed: Mar. 3, 2021
(86) PCT No.: PCT/IB2021/051783

§ 371 Date: Sep. 6, 2022

Related U.S. Application Data
(60) Provisional application No. 62/984,660, filed on Mar. 3, 2020.

(51) Int. Cl. A61L 27/40; B33Y 10/00; B33Y 80/00; A61L 27/36; A61L 27/56

(52) U.S. Cl. CPC A61L 27/40; B33Y 10/00; B33Y 80/00; A61L 27/3695; A61L 27/3645; A61L 27/56

(57) ABSTRACT

A mechanoactive material includes a composite textile that includes a textile substrate formed from a plurality of fibers assembled in a fiber assembly pattern and a material deposited via an additive manufacturing technique onto and/or between the fibers of the textile substrate based on an additive manufacturing pattern. The composite textile includes at least one prestress and/or residual stress and/or exhibits a change in at least one mechanical property, material property, or structure in response to at least one endogenous and/or exogenous stimulus.

### RELATED APPLICATION

This application claims priority from U.S. Provisional Application No. 62/984,660, filed Mar. 3, 2020, the subject matter of which is incorporated herein by reference in its entirety.

### BACKGROUND

It is desired to provide smarter materials for apparel, architecture, product design and manufacturing, aerospace and automotive industries. However, these capabilities have often required expensive, error-prone and complex electromechanical devices (e.g., motors, sensors, electronics), bulky components, power consumption (e.g., batteries or electricity) and difficult assembly processes. These constraints have made it challenging to efficiently produce dynamic systems, higher-performing machines and more adaptive products.

Further, while “smart” materials have been developed, which can provide some sort of a dynamic structure, such materials are often formed in fixed shapes and sizes. These materials must subsequently be assembled into the necessary end product form, typically using off the shelf (non-custom) parameters. These types of smart materials are extremely expensive and are generally only found in niche markets due to their cost. Further, using these smart materials to provide a specific type of product having a particular function requires significant skill and time.

### SUMMARY

Embodiments described herein relate to mechanoactive materials formed from composite textiles as well as to methods of forming mechanoactive materials and/or smart materials. The composite textiles can include a textile substrate formed from a plurality of fibers assembled in a fiber assembly pattern and a material deposited via an additive manufacturing technique onto and/or between fibers of the textile substrate based on an additive manufacturing pattern. The composite textile can include at least one prestress and/or residual stress and/or can exhibit a change in at least one mechanical property, material property, or structure in response to at least one endogenous and/or exogenous stimulus.

In some embodiments, prestresses and/or residual stresses in the composite textile can provide spatially and/or temporally resolved regions in the composite textile that can be used to sense, apply, transduce, release, and/or store at least one of forces, displacement and/or energy when triggered and/or activated.

In other embodiments, the fiber assembly pattern includes different biophysically responsive fibers in sections of the textile substrate. The different biophysically responsive fibers can provide at least one prestress and/or residual stress in the composite textile. The composite textile can include areas of prestresses and/or residual stress and/or bulk prestresses and/or residual stresses.

In other embodiments, at least one prestress and/or residual stress in the composite textile can be defined by at least one of the shape, material, and/or stiffness of the fibers. For example, the fiber assembly pattern can include selectively prestressed warp and weft fibers.

In some embodiments, the composite textile can include kirigami cut-outs and/or folding that provide at least one prestress and/or residual stress and/or gradient in at least one mechanical property, material property, or structural property of the composite textile.

In some embodiments, the mechanoactive material can act as at least one of a sensor, actuator, transducer, or capacitor in response to the endogenous and/or exogenous stimulus.

In other embodiments, the mechanoactive material can be a biomedical material, mechanically functional composite, absorbent article, drug delivery device, bioprosthetic device, biomaterial implant, microfluidic device, or vehicle occupant restraint.

In some embodiments, the fiber assembly pattern and/or the additive manufacturing pattern are based on an intrinsic pattern of at least one mechanical property, material property, or structural property of a biological material of interest. The biological material can include tissue of a plant, animal, or insect. For example, the fiber assembly pattern can be based on fingerprint patterns or geometric patterns with intrinsic elasticity and/or spatially distinct topography.

In other embodiments, the fiber assembly pattern can include a warp and weft that are interlaced, woven, knitted, and/or knotted to provide the at least one prestress and/or residual stress.

In some embodiments, the assembled fibers are woven using a weaving algorithm based on the intrinsic pattern to define the weave pattern and fiber orientation.

In some embodiments, the additive manufacturing technique comprises one or more of a fused deposition modeling (FDM) technique, a fused filament fabrication (FFF) technique, a big area additive manufacturing (BAAM) technique, a robocasting technique, an extrusion technique, an electrospinning technique, casting, and/or a direct ink writing (DIW) technique.

In other embodiments, the composite textile includes a plurality of the first regions spaced from one another in the composite textile and separated by second regions, wherein the first regions and second regions differ in at least one of a mechanical property, material property, or structural property.

In some embodiments, at least some of the first regions having a different porosity, volume, volumetric permeability, and/or surface permeability than the porosity, volume, volumetric permeability, and/or surface permeability of other first regions.

In other embodiments, the composite textile has a region of temporally-controlled elasticity that transitions between a first state and a second state in response to the external stimuli, wherein the first state is more relaxed than the second state, and the mechanoactive material can at least partially revert from the second state to the first state over an extended time period resulting from the temporally-controlled elasticity of the textile substrate.

In some embodiments, the internal energy of the mechanoactive material in the first state is less than internal energy of the substrate in the second state.

In other embodiments, different regions of the mechanoactive material possess different temporally-controlled elasticity.

In some embodiments, the mechanoactive material moves from the second state to the first state via any one of elongation or shortening of the mechanoactive material, or relaxation or stiffening of the mechanoactive material.

In some embodiments, the textile substrate possesses spatially-controlled elasticity, whereby different regions of the textile substrate have different elasticity.

In other embodiments, the textile substrate is woven using at least two threads/fibers, wherein each thread has a different elasticity.

In other embodiments, the textile substrate includes at least one thread possessing elasticity that varies along the length of the thread.

In other embodiments, the textile substrate includes at least one thread possessing elasticity that varies within the cross-section of the thread.

In still other embodiments, the textile substrate is woven using threads arranged in different directions such that the threads move frictionally relative to one another causing the transition from the first state to the second state to occur over an extended time period.

In other embodiments, the textile substrate can be woven using threads arranged in different directions such that the threads move frictionally relative to one another causing the transition from the first state to the second state to occur over an extended time period.

In some embodiments, the mechanoactive material or composite textile can further include at least one bioactive agent incorporated on or within the composite textile. The at least one bioactive agent can be capable modulating a function and/or characteristic of a cell. The bioactive material can include, for example, chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin-like growth factor (e.g., IGF-I, IGF-II) and transforming growth factors (e.g., TGF-β I-III)), parathyroid hormone, parathyroid hormone related peptide, bone morphogenic proteins (e.g., BMP-2, BMP-4, BMP-6, BMP-7, BMP-12, BMP-13, BMP-14), transcription factors, such as sonic hedgehog, growth differentiation factors (e.g., GDF5, GDF6, GDF8), recombinant human growth factors (e.g., MP52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, oligonucleotides, proteoglycans, glycoproteins, glycosaminoglycans, and DNA encoding for shRNA.

In other embodiments, the mechanoactive material or composite textile can include at least one cell dispersed on and/or within the composite textile. The cell can be, for example, a progenitor cell, such as totipotent stem cells, pluripotent stem cells, and multipotent stem cells, as well as any of their lineage descendant cells, including more differentiated cells. Exemplary progenitor cells can be selected from, but not restricted to, totipotent stem cells, pluripotent stem cells, multipotent stem cells, mesenchymal stem cells (MSCs), hematopoietic stem cells, neuronal stem cells, hematopoietic stem cells, pancreatic stem cells, cardiac stem cells, embryonic stem cells, embryonic germ cells, neural crest stem cells, kidney stem cells, hepatic stem cells, lung stem cells, hemangioblast cells, and endothelial progenitor cells. Additional exemplary progenitor cells are selected from, but not restricted to, de-differentiated chondrogenic cells, chondrogenic cells, cord blood stem cells, multi-potent adult progenitor cells, myogenic cells, osteogenic cells, tendogenic cells, ligamentogenic cells, adipogenic cells, and dermatogenic cells.

Other embodiments described herein relate to a method of forming a mechanoactive material. The method includes assembling a plurality of fibers based on a fiber assembly pattern into a textile substrate. A material is then deposited via an additive manufacturing technique onto and/or between fibers of the textile substrate based on an additive manufacturing pattern to provide a composite textile that includes at least one prestress and/or residual stress and/or exhibits a change in at least one mechanical property, material property, or structure in response to at least one endogenous and/or exogenous stimulus.

In some embodiments, prestresses and/or residual stresses in the composite textile provide spatially and/or temporally resolved regions to sense, apply, transduce, release, and/or store at least one of forces, displacement and/or energy when triggered and/or activated.

In other embodiments, the fiber assembly pattern includes different biophysically responsive fibers in sections of the textile substrate. The different biophysically responsive fibers can provide the at least one prestress and/or residual stress in the composite textile. The composite textile can include areas of prestresses and/or residual stress and/or bulk prestresses and/or residual stresses.

In other embodiments, the at least one prestress and/or residual stress in the composite textile is defined by at least one of the shape, material, and/or stiffness of the fibers. The fiber assembly pattern can include selectively prestressed warp and weft fibers.

In some embodiments, the method further includes cutting and/or folding the composite textile using a kirigami technique to generate two dimension and/or three dimensional cut patterns that provide at least one prestress and/or residual stress in the composite textile and/or gradient in at least one mechanical property, material property, or structural property of the composite textile.

In some embodiments, the method further includes mapping a three dimensional spatial distribution of at least one mechanical property, material property, or structure of a natural or biological material of interest and designing the fiber assembly pattern and/or the additive manufacturing pattern based the intrinsic pattern of at least one mechanical property, material property, or structural property of the biological material of interest.

In some embodiments, the fiber assembly pattern is based on fingerprint patterns or geometric patterns with intrinsic elasticity and/or spatially distinct topography.

In other embodiments, the fibers include a warp and weft that are interlaced, woven, knitted, and/or knotted into the fiber assembly pattern.

In some embodiments, the assembled fibers are woven using a weaving algorithm based on the intrinsic pattern to define the weave pattern and fiber orientation.

In other embodiments, the additive manufacturing technique comprises one or more of a fused deposition modeling (FDM) technique, a fused filament fabrication (FFF) technique, a big area additive manufacturing (BAAM) technique, a robocasting technique, a paste extrusion technique, casting technique, and/or a direct ink writing (DIW) technique.

In other embodiments, the composite textile includes a plurality of the first regions spaced from one another in the composite textile and separated by second regions, wherein the first regions and second regions differing in at least one of a mechanical property, material property, or structural property.

In some embodiments, at least some of the first regions having a different porosity, volume, volumetric permeability, and/or surface permeability than the porosity, volume, volumetric permeability, and/or surface permeability of other first regions.

In other embodiments, the composite textile has a region of temporally-controlled elasticity that transitions between a first state and a second state in response to the external stimuli, wherein the first state is more relaxed than the second state, and the mechanoactive material can at least partially revert from the second state to the first state over an extended time period resulting from the temporally-controlled elasticity of the textile substrate.

In some embodiments, the internal energy of the mechanoactive material in the first state is less than internal energy of the substrate in the second state.

In other embodiments, different regions of the mechanoactive material possess different temporally-controlled elasticity.

In some embodiments, the mechanoactive material moves from the second state to the first state via any one of elongation or shortening of the mechanoactive material, or relaxation or stiffening of the mechanoactive material.

In some embodiments, the textile substrate possesses spatially-controlled elasticity, whereby different regions of the textile substrate have different elasticity.

In other embodiments, the textile substrate is woven using at least two threads/fibers, wherein each thread has a different elasticity.

In other embodiments, the textile substrate includes at least one thread possessing elasticity that varies along the length of the thread.

In other embodiments, the textile substrate includes at least one thread possessing elasticity that varies within the cross-section of the thread.

In still other embodiments, the textile substrate is woven using threads arranged in different directions such that the threads move frictionally relative to one another causing the transition from the first state to the second state to occur over an extended time period.

In other embodiments, the textile substrate can be woven using threads arranged in different directions such that the threads move frictionally relative to one another causing the transition from the first state to the second state to occur over an extended time period.

### BRIEF DESCRIPTION OF THE DRAWINGS

FIGS. 1(A-F) illustrates imaging based studies to elucidate multi-time and length scale properties of tissues, using bone and skin-on-bones (periosteum) as examples. Transitions between Epithelial Sheets (A) and Mesenchyme (B) occur throughout life, providing tissue geometries for functional boundaries and tissue structures. (C) At every length scale, mechanical forces are transduced to and sensed by cells, which serve as actuators to build structural proteins that confer toughness, elasticity and turgor to tissue constructs. (C′) After exposure to subtle cues such as 0.8 Pa fluid flow, cells buttress themselves by adapting their cytoskeleton (actin in red) and manufacturing structural proteins that are secreted to the extracellular matrix (collagen autofluorescing in green), thus modulating the cell's own local environment. Scalebar: 5 μm. (D+) Once collagens are fully assembled in the extracellular matrix, they resemble curled or crimped structures which provide a scaffold for adherence of quiescent stem cells. Here, in the matrix of the periosteum, the tissue is tacked on to bony surfaces through an abundance of Sharpey's fibers which help the tissue maintain a state of prestress. (D−) Once these fibers are cut or disturbed through periosteal lifting surgery or trauma, the prestress in the tissue relaxes; (see graph at right) and the collagen crimp increases significantly when prestress is removed, resulting in rounding of cells, a hypothetical trigger for stem cell egression to sites of injury. In this way, the “brainless” cells and the matrix interact as smart sensors and transducers and actuators. (E) When administered at known points in time, mineral chelating fluorochromes mark areas of mineralization which can be decoded to show temporal dynamics of mineralization under specific excitation and emission frequencies of light. Scalebar: 50 μm. (F) Mineral nucleates around collagens. The collagens (yellow) and elastins (green) making up the tissue demonstrate the textile structure of tissues (muscle fascicles in lower left quadrant with bone weave in upper right quadrant, and periosteum in between). Together with the organic and inorganic extracellular matrix components, the tissue represents a complex composite structure. Scalebar: 50 μm. Figure adapted and compiled from previously published figures, with permission.

FIGS. 2(A-H) illustrate smart properties of mechanoactive materials, including skin-on-bones (periosteum, A,B,C,E) and bone itself (D) and MADAME approaches to emulate those properties (F,G1-G4,H1-H4). (A) Micron-resolution strain mapping of periosteum in situ under stance-shift loading demonstrates surprising heterogeneity. (B1-B4) Similar heterogeneity is observed in strains during tensile testing of periosteum samples taken in the Axial and Circumferential directions (C) of the anterior (front) femur, which exhibit significant anisotropy. (C—Axial) Specimens taken along the length of the femur exhibit strain stiffening while those taken along the circumference (C—Circumferential) of the femur exhibit linear elasticity. Removal of the samples through cutting of the Sharpey's fibers and sample edges results in grossly observable shrinking of the specimens, which is significantly greater along the length of the long bone (Axial) compared to its circumference. Based on shrinkage and measured moduli of elasticity, prestress of the tissue in situ can be calculated. (D) Bone shows several calibers of porosity, including predominantly porosity around osteocytes (lacunar) and blood vessels (vascular). When the density of lacunar and vascular porosities are depicted spatially and as a heat map with warmer colors depicting higher density, the different patterns of lacunar (D1) and vascular (D2) densities are striking. Experimentally based computational models reveal counterintuitive flow effects when one explicitly accounts for these different spatial patterns of porosity densities, e.g., exudation of fluid under tension and imbibement of fluid under compression, which is contrary to daily experience with wet kitchen sponges. (E1,E2) Permeability studies on periosteum show a strong increase in permeability when prestress is removed from the tissue through cutting of Sharpey's fibers. In addition, permeability depends strongly on volumetric flow rate and direction of flow, where flow in the bone to muscle direction increases significantly more than that in the opposite direction with increase in volumetric flow rate; p<0.05 defines significant differences, * within, and ** between groups. (F) Mechanical patterns resulting from intrinsic tissue weaves of e.g., elastin and collagen can be recreated recursively to create textiles emulating strain patterns observed in tissues. In this case, color patterns represent patterns of strain or fiber stiffness. Conceptually, “unraveling” of the tissue results in a singular solution with a fiber of varying stiffness along its length. An infinite number of solutions can be achieved through algorithms encoding patterns of stiffness, where microscopy acquired images (G1) provide a basis for infinite digital patterns and virtual prototypes (G2) that can be turned into physical prototypes using computer-controlled textile and knit systems, alone and/or in combination with other advanced manufacturing modalities such as multidimensional printing, laser sintering, etc. As a whole, the process is referred to as Microscopy-Aided Design And ManufacturE. Figure adapted and compiled from previously published figures, with permission.

FIGS. 3(A-C) illustrates a concept of encoding mechanical phenotype in synthetic polymeric materials and a recursive logic approach to encoding mechanoactive material properties in textile (and composite) patterns comprising fibers of different mechanical phenotypes. (A) Thermoplastics, synthetic elastomers and gels and biological networks (extracellular matrix, tissue) exhibit mechanical phenotypes exemplified through the materials' stress σ-strain ε curve (EO, the Young's Modulus, is the material stiffness in the linear elastic region of the curve; β shows strain stiffening or non-linear increase in Modulus with increase in deformation, a measure of material firmness; 94 max is the strength of the material or stress at break; the area under the curve indicates material toughness or how much energy is dissipated at breakage. (B) Elongation at break (λmax) versus Young's modulus (E) over the mass density (ρ) of a material. “This representation recovers scaling relations E˜λmax-2, for elastomeric materials with λmax»1, and E˜εmax-2, for hard materials with strain at break εmax=λmax-1«1.” (C) Master Table of properties for materials created using MADAME and tested in tension, compared to textile and isotropic control materials.

FIG. 4 illustrates schematics of mechanoactive materials having fingerprint patterns or coiled patterns.

FIG. 5 illustrates a schematic of a mechanoactive material that includes multiple sections with coiled patterns that include fibers and/or fiber assembly patterns with elasticity that varies along their length.

FIG. 6 illustrates a schematic of mechanoactive material having a fingerprint pattern that include fibers and/or fiber assembly patterns with elasticity that varies along their length.

### DETAILED DESCRIPTION

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law tradition, the terms “a”, “an”, and “the” are meant to refer to one or more as used herein, including the claims. For example, the phrase “a cell” can refer to one or more cells.

The term “absorbable” is meant to refer to a material that tends to be absorbed by a biological system into which it is implanted. Representative absorbable fiber materials include, but are not limited to polyglycolic acid (PGA), polylactic acid (PLA), polyglycolide-lactide, polycaprolactone, polydioxanone, polyoxalate, a polyanhydride, a poly(phosphoester), catgut suture, collagen, silk, chitin, chitosan, hydroxyapatite, bioabsorbable calcium phosphate, hyaluronic acid, and any other medically acceptable yet absorbable fiber. Other absorbable materials include collagen, gelatin, a blood derivative, plasma, synovial fluid, serum, fibrin, hyaluronic acid, a proteoglycan, elastin, and combinations thereof.

The term “non-absorbable” is meant to refer to a material that tends not to be absorbed by a biological system into which it is implanted. Representative non-absorbable fiber materials include but are not limited to polypropylene, polyester, polytetrafluoroethylene (PTFE) such as that sold under the registered trademark TEFLON (E.I. DuPont de Nemours & Co., Wilmington, Del., United States of America), expanded PTFE (ePTFE), polyethylene, polyurethane, polyamide, nylon, polyetheretherketone (PEEK), polysulfone, a cellulosic, fiberglass, an acrylic, tantalum, polyvinyl alcohol, carbon, ceramic, a metal (e.g., titanium, stainless steel), and any other medically acceptable yet non-absorbable fiber.

The terms “anisotropic”, “anisotropy”, and grammatical variations thereof, refer to properties of a textile, composite, and/or fiber system as disclosed herein that can vary along a particular direction. Thus, the fiber, composite, and/or textile can be stronger and/or stiffer in one direction versus another. In some embodiments, this can be accomplished by changing fibers (such as, but not limited to providing fibers of different materials) in warp versus weft directions, and/or in the Z direction, for example, or changing the material disposed using the additive manufacturing technique.

The terms “anisotropic”, “anisotropy” and grammatical variations thereof, can also include, but is not limited to the provision of more fiber or disposed material in a predetermined direction. This can thus include a change of diameter in a fiber over a length of the fiber, a change in diameter at each end of the fiber, and/or a change in diameter at any point or section of the fiber; a change in cross-sectional shape of the fiber; a change in density or number of fibers in a volumetric section of the scaffold; the use of monofilament fibers and/or multifilament fibers in a volumetric section of the textile, or the use of different types, amounts, or densities of deposited materials; and can even include the variation in material from fiber system to fiber system and along individual fibers in a volumetric section of the textile.

The terms “biocompatible” and “medically acceptable” are used synonymously herein and are meant to refer to a material that is compatible with a biological system, such as that of a subject having a tissue to be repaired, restored, and/or replaced. Thus, the term “biocompatible” is meant to refer to a material that can be implanted internally in a subject as described herein.

The term “composite material”, as used herein, is meant to refer to any material comprising two or more components.

The term “bioactive agent” can refer to any agent capable of promoting tissue formation, destruction, and/or targeting a specific disease state (e.g., cancer). Examples of bioactive agents can include, but are not limited to, chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin-like growth factor (e.g., IGF-I, IGF-II) and transforming growth factors (e.g., TGF-β I-III)), parathyroid hormone, parathyroid hormone related peptide, bone morphogenic proteins (e.g., BMP-2, BMP-4, BMP-6, BMP-7, BMP-12, BMP-13, BMP-14), transcription factors, such as sonic hedgehog, growth differentiation factors (e.g., GDF5, GDF6, GDF8), recombinant human growth factors (e.g., MP52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, oligonucleotides, proteoglycans, glycoproteins, glycosaminoglycans, and DNA encoding for shRNA.

The term “bioresorbable” can refer to the ability of a material to be fully resorbed in vivo. “Full” can mean that no significant extracellular fragments remain The resorption process can involve elimination of the original implant material(s) through the action of body fluids, enzymes, cells, and the like.

The term “cell” can refer to any progenitor cell, such as totipotent stem cells, pluripotent stem cells, and multipotent stem cells, as well as any of their lineage descendant cells, including more differentiated cells. The terms “stem cell” and “progenitor cell” are used interchangeably herein. The cells can derive from embryonic, fetal, or adult tissues. Exemplary progenitor cells can be selected from, but not restricted to, totipotent stem cells, pluripotent stem cells, multipotent stem cells, mesenchymal stem cells (MSCs), hematopoietic stem cells, neuronal stem cells, hematopoietic stem cells, pancreatic stem cells, cardiac stem cells, embryonic stem cells, embryonic germ cells, neural crest stem cells, kidney stem cells, hepatic stem cells, lung stem cells, hemangioblast cells, and endothelial progenitor cells. Additional exemplary progenitor cells are selected from, but not restricted to, de-differentiated chondrogenic cells, chondrogenic cells, cord blood stem cells, multi-potent adult progenitor cells, myogenic cells, osteogenic cells, tendogenic cells, ligamentogenic cells, adipogenic cells, and dermatogenic cells.

The term “effective amount” refers to an amount of a bioactive agent sufficient to produce a measurable response (e.g., a biologically relevant response in a cell exposed to the differentiation-inducing agent) in the cell. In some embodiments, an effective amount of a differentiation-inducing agent is an amount sufficient to cause a precursor cell to differentiate in in vitro culture into a cell of a tissue at predetermined site of treatment. It is understood that an “effective amount” can vary depending on various conditions including, but not limited to the stage of differentiation of the precursor cell, the origin of the precursor cell, and the culture conditions.

The terms “inhomogeneous”, “inhomogeneity”, “heterogeneous”, “heterogeneity”, and grammatical variations thereof, are meant to refer to a fiber, substrate, textile, composite, and/or fabric as disclosed herein that does not have a homogeneous composition along a given length or in a given volumetric section. In some embodiments, an inhomogeneous construct as disclosed herein comprises a composite material, such as a composite comprising a three dimensional woven fiber substrate, textile, and/or fabric as disclosed herein, cells that can develop tissues that substantially provide the function of periosteum, cartilage, other tissues, or combinations thereof, and a matrix that supports the cells. In some embodiments, an inhomogeneous substrate as disclosed herein can comprise one or more component systems that vary in their properties according to a predetermined profile, such as a profile associated with the tissue and/or other location in a subject where the substrate will be implanted. Thus, it is an aspect of the terms “inhomogeneous”, “inhomogeneity”, “heterogeneous”, “heterogeneity”, and grammatical variations thereof to encompass the control of individual materials and properties in the substrate.

The terms “non-linear”, “non-linearity”, and grammatical variations thereof, refer to a characteristic provided by a fiber substrate, textile, and/or fabric as disclosed herein such that the fiber substrate, textile, and/or fabric can vary in response to a strain. Fiber substrate, textile, and/or fabric disclosed herein can provide stress/stain profiles that mimic that observed in a target or region of interest.

The terms “resin”, “matrix”, or “gel” are used in the art-recognized sense and refer to any natural or synthetic solid, liquid, and/or colloidal material that has characteristics suitable for use in accordance with the presently disclosed subject matter. Representative “resin”, “matrix”, or “gel” materials thus comprise biocompatible materials. In some embodiments, the “resin”, “matrix”, or “gel” can occupy the pore space of a textile substrate as disclosed herein.

The term “smart material(s)” refers to a designed material that have one or more properties that can be changed in a controlled fashion under the influence of an external stimulus, such as stress, temperature, moisture, pH, electric or magnetic fields. This change can be reversible and can be repeated many times.

The term “sensor” refers to a device, module, or system that senses shifts in energy, force, strain, displacements between the object/point of sensor contact and its interface with its environment, via changes in e.g. electrical resistance. Examples include tactile sensors, pressure sensors, strain sensors, temperature, capacitive and elastoresistive sensors.

The term “actuator” refers to a device, module, or system that applies forces or displacements, effecting work.

The term “transducer” refers to a device, module, or system that converts one form of energy to another, e.g. transduces forces or displacements or strain into electrical signals.

The term “capacitor” refers to a device, module, or system that stores and releases energy.

As used herein, “structural material” means a material used in constructing a wearable, personal accessory, luggage, etc. Examples of structural materials include: fabrics and textiles, such as cotton, silk, wool, nylon, rayon, synthetics, flannel, linen, polyester, woven or blends of such fabrics, etc.; leather; suede; pliable metallic such as foil; Kevlar, etc. Examples of wearables include: clothing; footwear; prosthetics such as artificial limbs; headwear such as hats and helmets; athletic equipment worn on the body; protective equipment such as ballistic vests, helmets, and other body armor. Personal accessories include: eyeglasses; neckties and scarfs; belts and suspenders; jewelry such as bracelets, necklaces, and watches (including watch bands and straps); and wallets, billfolds, luggage tags, etc. Luggage includes: handbags, purses, travel bags, suitcases, backpacks, and including handles for such articles, etc.

The terms “viscoelastic”, “viscoelasticity”, and grammatical variations thereof, are meant to refer to a characteristic provided by a fiber substrate, textile, and/or fabric as disclosed herein that can vary with a time and/or rate of loading.

Embodiments described herein relate to mechanoactive materials formed from composite textiles as well as to methods of forming mechanoactive materials and/or smart materials. The composite textile can include a textile substrate formed from a plurality of fibers assembled in a fiber assembly pattern and a material deposited via an additive manufacturing technique onto and/or between fibers of the textile substrate based on an additive manufacturing pattern. The composite textile can include at least one prestress and/or residual stress and/or can exhibit a change in at least one mechanical property, material property, or structure in response to at least one endogenous and/or exogenous stimulus.

The mechanoactive textiles and/or smart materials formed therefrom can act as sensors, actuators, and transducers, with inductive and/or capacitive function. The materials can achieve this through patterns and higher order architectures that include different biophysically responsive fibers in space, and/or kirigami cut outs that substitute for warp or weft fibers in sections of the textile, as well as the fiber patterns'/architectures'/cut outs' unique spatial and temporal responses to exogenous and endogenous stimuli.

In some embodiments, the prestresses and/or residual stresses in the composite textile can provide spatially and/or temporally resolved regions to sense, apply, transduce, release, and/or store at least one of forces, displacement and/or energy when triggered and/or activated. Such stresses can be achieved through, for example: kirigami and fiber shape, material, and/or stiffness selection; hollow fibers filled with different phase material (nano-microfluidics) and/or cloaking material (insulated wires) with different biophysical properties; regional differences in mechanoactivation/sensing/transducing and capacitance function; and/or sacrificial struts, fibers, and/or sheaths.

The composite textile can also include a gradient in least one of mechanical property (e.g., tension, compression, elasticity, stiffness, density, hardness, strength, toughness, etc.), material property (e.g., degradability, reactivity) , or structural property (e.g., shape, porosity, permeability, etc.) and/or exhibit a change in at least one mechanical property, material property, or structure in response to at least one external to at least one endogenous and/or exogenous stimulus (e.g., stress, temperature, moisture, pH, electric or magnetic fields, etc.).

In some embodiments, the engineered composite textiles can replicate or mimic biological or natural material's or nature's intrinsic architecture of structural molecules by translation of nature's intrinsic architecture to weave scaled-up, multidimensional composite textile architectures emulating natural material organization. The methods and composite textiles described herein can provide mechanically functional textiles, including but not limited engineered tissue fabrics and tissue implants, and materials for transport and safety industries, structural material, biomedical materials, absorbent articles, drug delivery devices, bioprosthetic devices, biomaterial implants, flooring, transport safety devices (seat belts, airbags and auto safety nets for autonomous vehicles of the future), microfluidic devices, remote applications (haptics, robotics, remote healthcare), bionics (active restoration of function), and/or functional augmentation (powered exoskeletons for rescue, toxic environments, injury prevention).

In some embodiments, a method of forming a mechanoactive material that includes a composite textile, such as a biomedical material, tissue implant, or mechanically functional textile, can include assembling a plurality of fibers based on a fiber assembly pattern into a textile substrate and depositing a material via an additive manufacturing technique between and/or onto fibers of the textile substrate based on an additive manufacturing pattern to provide a composite textile that can include at least one prestress and/or residual stress and/or exhibits a change in at least one mechanical property, material property, or structure in response to at least one endogenous and/or exogenous stimulus.

In some embodiments, the method can further include mapping a three dimensional spatial distribution of at least one mechanical property, material property, or structure of a natural or biological material of interest. For example, the fiber assembly pattern can be designed based on an intrinsic pattern of at least one structural molecule of a natural material or a biological material.

In some embodiments, the biological material can include tissue of a plant, animal, or insect. The tissue can include, for example, at least one or periosteum, pericardium, perimycium, or tissue bounding an organ or tissue compartment (e.g., tree bark). In another example, the fiber assembly pattern can be based on fingerprint patterns or geometric patterns with intrinsic elasticity and/or spatially distinct topography.

Regions of interest (ROI) of the natural or biological material can be imaged and mapped to highlight gradients of mechanical properties, material properties, or structure of a natural or biological material. For example, ROI in context of tissue compartments (bone, muscle, vasculature) and their respective microscopic structures can be mapped along the major and minor axes. These axes, calculated using an automated software, can serve, for example, as objective indicators of tissue regions most and least able to resist bending forces in the axial plane. For each ROI, a tiled image of the transverse (xy) plane, followed by a z-stack of one tile within the region, can be captured to map in 3D space the composition and distribution of structural molecules, such as collagen and elastin fibers, as well as their higher order architectures.

In some embodiments, the three dimensional spatial distribution biological material or patterns can be mapped or imaged using multimodal imaging of section or transverse section of a biological material. For example, the three dimensional spatial distribution of the collagen fibers and the elastin fibers can be mapped using, respectively, second harmonic imaging microscopy and two photon excitation imaging microscopy of transverse section of ROI of the biological material.

Second harmonic imaging microscopy (SHIM) can be used to capture high-resolution, high-content, 3D representations of fibrillar collagen in live and ex vivo tissue without the need for exogenous labeling. In SHIM, a frequency doubling of the incident light occurs in repetitive and non-centrosymmetric molecular structures.

By way of example, biological specimens can be imaged using a Leica SP5 II inverted microscope equipped with a Spectra Physics MaiTai HP DeepSea titanium sapphire multiphoton laser tuned to 830 nm (˜100 fs pulse), a xyz high precision multipoint positioning stage and a 63× 1.3NA glycerol objective. The forward propagated second harmonic collagen signal can then be collected in the transmitted Non-Descanned-Detector using a 390-440 nm bandpass filter.

The two-photon imaging of elastin can be performed by excitation of the biological specimen at 830 nm and following by collection using a photo-multiplier tube (PMT) with a 435-495 nm emission filter. This filter can be used to segment away autofluorescence.

The images can then collated to create to create scaled up three dimensional maps or models, which accurately represent the composition and spatial architecture of the image sequences and the biological material and/or tissue itself. The three dimensional maps can include not only the spatial distribution of the structural molecules, but also other features or structures including topography and vasculature that extends through the matrix.

Following mapping of the three dimensional spatial distribution of at least one mechanical property, material property, or structure of a natural or biological material a fiber assembly pattern can be designed based on an intrinsic pattern of the mapped mechanical property, material property, or structural property. In some embodiments, the fiber assembly pattern can include a weaving algorithm or weaving motif based on the intrinsic pattern of the mapped three dimensional spatial distribution mechanical property, material property, or structural property as well as other structural features. In some instances, the intrinsic pattern of the can be used to design or generate a custom-configured jacquard weaving algorithm (ArahWeave, arahne CAD/CAM for weaving) for weaving of physical prototypes (AVL Looms, Inc.).

Following design of the fiber assembly pattern, fibers can be woven in a weave pattern and/or fiber orientation based on the fiber assembly pattern or weaving algorithm to form a textile substrate. The fibers woven using the weaving algorithm can be monofilament, multifilament, or a combination thereof, and can be of any shape or cross-section including, but not limited to bracket-shaped (i.e., [), polygonal, square, I-beam, inverted T shaped, or other suitable shape or cross-section. The cross-section can vary along the length of fiber. Fibers can also be hollow to serve as a carrier for bioactive agents (e.g., antibiotics, growth factors, etc.), cells, and/or other materials as described herein. In some embodiments, the fibers can serve as a degradable or non-degradable carriers to deliver a specific sequence of growth factors, antibiotics, or cytokines, etc., embedded within the fiber material, attached to the fiber surface, or carried within a hollow fiber. The fibers can each comprise a biocompatible material, and the biocompatible material can comprise an absorbable material, a non-absorbable material, or combinations thereof.

Fiber diameters can be of any suitable length in accordance with characteristics composite textile's use or function. Representative size ranges include a diameter of about 1 micron, about 5 microns, about 10 microns about 20 microns, about 40 microns, about 60 microns, about 80 microns, about 100 microns, about 120 microns, about 140 microns, about 160 microns, about 180 microns, about 200 microns, about 220 microns, about 240 microns, about 260 microns, about 280 microns, about 300 microns, about 320 microns, about 340 microns, about 360 microns, about 380 microns, about 400 microns, about 450 microns or about 500 microns (including intermediate lengths). In various embodiments, the diameter of the fibers can be less than about 1 micron or greater than about 500 microns. Additionally, nanofibers fibers with diameters in the nanometer range (1-1000 nanometers) are envisioned for certain embodiments. Additionally, large fibers with diameters up to 3.5 cm are envisioned for certain embodiments.

In other embodiments, the fibers or subset of fibers, can contain one or more bioactive or therapeutic agents such that the concentration of the bioactive or therapeutic agent or agents varies along the longitudinal axis of the fibers or subset of fibers. The concentration of the active agent or agents can vary linearly, exponentially or in any desired fashion, as a function of distance along the longitudinal axis of a fiber. The variation can be monodirectional; that is, the content of one or more therapeutic agents can decrease from the first end of the fibers or subset of the fibers to the second end of the fibers or subset of the fibers. The content can also vary in a bidirectional fashion; that is, the content of the therapeutic agent or agents can increase from the first ends of the fibers or subset of the fibers to a maximum and then decrease towards the second ends of the fibers or subset of the fibers.

Thus, in some embodiments, the fibers serve as a degradable or nondegradable carrier to deliver one or more specific sequences of growth factors, antibiotics, cytokines, etc. that are embedded within the fiber matter, attached to the fiber surface, or carried within a hollow fiber.

In some embodiments, the fibers woven to form the textile substrate can be prepared in a hydrated form or it can be dried or lyophilized into a substantially anhydrous form.

In other embodiments, the fibers can be biodegradable over time, such that it will be absorbed into a subject if implanted in a subject. Woven fiber substrates, which are biodegradable, can be formed from monomers, such as glycolic acid, lactic acid, propyl fumarate, caprolactone, hyaluronan, hyaluronic acid, and the like. Other fiber substrates can include proteins, polysaccharides, polyhydroxy acids, polyorthoesthers, polyanhydrides, polyphosazenes, or synthetic polymers (particularly biodegradable polymers). In some embodiments, polymers for forming the fiber substrates can include more than one monomer (e.g., combinations of the indicated monomers). Further, the fiber substrate can include hormones, such as growth factors, cytokines, and morphogens (e.g., retinoic acid, arachidonic acid, etc.), desired extracellular matrix molecules (e.g., fibronectin, laminin, collagen, etc.), or other materials (e.g., DNA, viruses, other cell types, etc.) as desired.

Polymers used to form the fibers can include single polymer, co-polymer or a blend of polymers of poly(L-lactic acid), poly(DL-lactic acid), polycaprolactone, poly(glycolic acid) or polyanhydride. Naturally occurring polymers can also be used such as reconstituted or natural collagens or silks. Those of skill in the art will understand that these polymers are just examples of a class of biodegradable polymers that can be used in the presently disclosed subject matter. Further biodegradable polymers include polyanhydrides, polyorthoesters, and poly(amino acids).

Examples of natural polymers that can be used for the fibers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, glucose, polyglucose, polydextrose, pustulan, chitin, agarose, keratin, chondroitin, dermatan, hyaluronic acid, alginic acid, xanthan gum, starch and various other natural homopolymer or heteropolymers, such as those containing one or more of the following aldoses, ketoses, acids or amines: erythrose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Accordingly, suitable polymers can include, for example, proteins, such as albumin.

Examples of semi-synthetic polymers that can be used to form the fibers include carboxymethylcellulose, hydroxymethylcellulose, hydroxypropylmethylcellulose, methylcellulose, and methoxycellulose. Exemplary synthetic polymers include polyphosphazenes, polyethylenes (such as, for example, polyethylene glycol (including the class of compounds referred to as PLURONICS, commercially available from BASF, Parsippany, N.J., U.S.A.), polyoxyethylene, and polyethylene terephthlate), polypropylenes (such as, for example, polypropylene glycol), polyurethanes, polyvinyl alcohol (PVA), polyvinyl chloride and polyvinylpyrrolidone, polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof.

In some embodiments, the fibers can be assembled into a three dimensional fiber substrate, or textile substrate using a 3-D computer controlled weaving loom, such as a jacquard loom, specifically constructed to produce precise structures from fine diameter fibers. The weaving pattern of the woven substrate, textile, or fabric is defined by the fiber assembly pattern or weaving algorithm designed from the intrinsic pattern of the mapped mechanical properties, material properties, structural molecules, e.g., structural protein fibers of the extracellular matrix of the biologic material.

The weaving pattern and/or weaving algorithm can also use or incorporate spatial and temporal patterns of (in-)elasticity to create dynamic pressures, such as described in WO2015/021503. The textile at least one region of temporally-controlled elasticity may include a step of weaving threads having varying composition and/or elasticity along their length into the substrate.

In some embodiments, the fiber assembly pattern includes different biophysically responsive fibers in sections of the textile substrate. The different biophysically responsive fibers can provide at least one prestress and/or residual stress in the composite textile. The composite textile can include areas of prestresses and/or residual stress and/or bulk prestresses and/or residual stresses.

In other embodiments, at least one prestress and/or residual stress in the composite textile is defined by at least one of the shape, material, and/or stiffness of the fibers. For example, the fiber assembly pattern can include selectively prestressed warp and weft fibers.

In some embodiments, the textile substrate and/or composite textile formed therefrom can have a region of temporally-controlled elasticity that transitions between a first state and a second state in response to the external stimuli. The first state can be more relaxed than the second state, and the smart material can at least partially revert from the second state to the first state over an extended time period resulting from the temporally-controlled elasticity of the textile substrate. The internal energy of the smart material in the first state can be less than internal energy of the substrate in the second state. Different regions of the smart material can possess different temporally-controlled elasticity.

In some embodiments, the textile substrate and/or composite textile formed therefrom can move from the second state to the first state via any one of elongation or shortening of the smart material, or relaxation or stiffening of the smart material. The textile substrate and/or composite textile formed therefrom can possess spatially-controlled elasticity, whereby different regions of the textile substrate have different elasticity or stiffness.

In some embodiments, the textile substrate can be woven using at least two threads/fibers, wherein each thread has a different elasticity.

In other embodiments, the textile substrate can include at least one thread possessing elasticity that varies along the length of the thread. The textile substrate can include at least one thread possessing elasticity that varies within the cross-section of the thread.

In other embodiments, the textile substrate can be woven using threads arranged in different directions such that the threads move frictionally relative to one another causing the transition from the first state to the second state to occur over an extended time period.

In other embodiments, the fiber assembly pattern includes a warp and weft that are interlaced, woven, knitted, and/or knotted to provide the at least one prestress and/or residual stress.

A computer controlled weaving machine can produce true 3-D shapes by placing fibers axially (x-warp direction), transversely (y-weft, or filling direction), and vertically (z-thickness direction). Multiple layers of warp yarns are separated from each other at distances that allow the insertion of the weft layers between them. Two layers of Z-yarns, which are normally arranged in the warp direction, are moved (after the weft insertion) up and down, in directions opposite to the other. This action is followed by the “beat-up”, or packing of the weft into the scaffold, and locks the two planar fibers (the warp and weft) together into a uniform configuration. Change of yarn densities can be achieved for warp by altering the reed density and warp arrangement and for weft by varying the computer program controlling the take-up speed of a stepper motor.

An advantage of the presently disclosed weaving technique is that each fiber can be selected individually and woven into a textile substrate. Using this method of assembly, customized structures can be easily created by selectively placing different constituent fibers (e.g., fibers of various material composition, size, and/or coating/treatment) throughout the textile substrate. In this manner, physical and mechanical properties of the textile substrate can be controlled (i.e., pore sizes can be selected, directional properties can be varied, and discreet layers can be formed). Using this technique, the inhomogeneity and anisotropy of various tissues can be reproduced by constructing a textile substrate that mimics the normal stratified structural network using a single, integral textile substrate.

In some embodiments, the fibers can be provided as threads that are oriented in space relative to each other during the assembly step. The assembly step includes can including orienting threads having different elasticity along their length according to a predetermined algorithm.

In other embodiments, yarns of the fibers after assembly can be set via any of a number of art-recognized techniques, including but not limited to ultrasonication, a resin, infrared irradiation, heat, or any combination thereof. Setting of the yarn systems within the scaffold in this manner provides cuttability and suturability. Sterilization can be performed by routine methods including, but not limited to autoclaving, radiation treatment, hydrogen peroxide treatment, ethylene oxide treatment, and the like.

Representative methods for making three-dimensional textile substrates are also disclosed in U.S. Pat. Nos. 5,465,760 and 5,085,252, the contents of each of which are incorporated herein by reference in their entireties. The following patent publications are also incorporated herein by reference in their entireties: PCT International Patent Application Publication WO 01/38662 (published May 31, 2001); PCT International Patent Application Publication WO 02/07961 (published Jan. 31, 2002); U.S. Patent Application Publication 2003/0003135 (published Jan. 2, 2003), and PCT International Patent Application Serial No. PCT/US06/14437, filed Apr. 18, 2006.

Following or during formation of the textile substrate, a material can deposited via an additive manufacturing technique onto and/or between the fibers of the textile substrate based on an additive manufacturing pattern to form a composite textile that includes at least one prestress and/or residual stress and/or exhibits a change in at least one mechanical property, material property, or structure in response to at least one endogenous and/or exogenous stimulus.

The material deposited via the additive manufacturing technique can include any known inorganic or organic material that can be deposited using additive manufacturing techniques. Such materials can include, for example, plastics or polymers, epoxies, elastomers, reactive polymer systems (e.g., polyurethane, polyurea), preceramic polymer resins, ceramics, metals, bio-materials, gels, and/or inks

In some embodiments, the plastics or polymers can include aliphatic, polycarbonate based thermoplastic polyurethanes, thermoplastic elastomers, polytetramethylene glycol based polyurethane elastomers, polyethylene naphthalate and isomers thereof, such as 2,6-, 1,4-, 1,5-, 2,7-, and 2,3-polyethylene naphthalate; polyalkylene terephthalates such as polyethylene terephthalate, polybutylene terephthalate, and poly-1,4-cyclohexanedimethylene terephthalate; aromatic polyesters, polyimides, such as polyacrylic imides; polyetherimides; styrenic polymers, such as atactic, isotactic and syndiotactic polystyrene, α-methyl-polystyrene, para-methyl-polystyrene; polycarbonates such as bisphenol-A-polycarbonate (PC); poly(meth)acrylates such as glassy poly(methyl methacrylate), poly(methyl methacrylate), poly(isobutyl methacrylate), poly(propyl methacrylate), poly(ethyl methacrylate), poly(butyl acrylate) and poly(methyl acrylate) (the term “(meth)acrylate” is used herein to denote acrylate or methacrylate); cellulose derivatives, such as ethyl cellulose, cellulose acetate, cellulose propionate, cellulose acetate butyrate, and cellulose nitrate; polyalkylene polymers, such as polyethylene, polypropylene, polybutylene, polyisobutylene, and poly(4-methyl)pentene; fluorinated polymers, such as perfluoroalkoxy resins, polytetrafluoroethylene, fluorinated ethylene-propylene copolymers, polyvinylidene fluoride, and polychlorotrifluoroethylene and copolymers thereof; chlorinated polymers, such as polydichlorostyrene, polyvinylidene chloride and polyvinylchloride; polysulfones; polyethersulfones; polyacrylonitrile; polyamides; polyvinylacetate; aromatic polyamides (e.g., amorphous nylons, such as Dupont Sellar or EMS G21), and polyether-amides as well as natural polymer macromers, such as poly(saccharide), poly(HEMA), collagen, fibrin, gelatin, glycosaminoglycans (GAG), poly (hyaluronic acid), poly(sodium alginate), alginate, hyaluronan, agarose, copolymers thereof, and blends thereof.

Examples of inorganic materials include metal, semiconductor, and or non-metal materials, such as bismuth ferrite (BiFeO₃), cadmium sulfide (CdS), cadmium telluride (CdTe), fullerenes (C60), graphite, graphene oxide, carbon nanoparticles, zinc oxide (ZnO) titanium dioxide (TiO₂) particles, metal particles, metal coated particles, inorganic oxides, metal oxides, and combinations thereof.

The material can be deposited onto and/or between fibers of the textile substrate using any additive manufacturing technique based on the additive manufacturing pattern. The additive manufacturing technique can include, for example, one or more of a fused deposition modeling (FDM) technique, a fused filament fabrication (FFF) technique, a big area additive manufacturing (BAAM) technique, a robocasting technique, an electrospinning technique, an extrusion technique, casting technique, and/or a direct ink writing (DIW) technique.

In one example, the material can be deposited onto and/or between fibers of the textile substrate using 3D printing. 3D printing has conventionally been used to create static objects and other stable structures, such as prototypes, products, and molds. Three-dimensional printers can convert a 3D image, which is typically created with computer-aided design (CAD) software, into a 3D object through the layer-wise addition of material.

One example of such a 3D printing technology includes multi-material three-dimensional (3D) printing technologies, which allow for deposition of material patterns with heterogeneous composition. For example, 3D printed structures can be composed of two or more materials having particular physical and chemical properties. Examples of 3D printers that can be used for the 3D printing of multi-material objects are described in U.S. Pat. Nos. 6,569,373; 7,225,045; 7,300,619; and 7,500,846; and U.S. Patent Application Publication Nos. 2013/0073068 and 2013/0040091, each of the teachings of which being incorporated herein by reference in their entireties. Printing of materials having a variety of properties, including rigid and soft plastics and transparent materials, and provide high-resolution control over material deposition. One of skill in the art will understand that it may be necessary to cure (e.g., polymerize) the 3D printed material.

The additive manufacturing pattern used for printing of the material can be designed by reference to a predetermined 3D geometric shape. In some embodiments, the additive manufacturing pattern can be based on the mapped three dimensional spatial distribution of at least one mechanical property, material property, or structure of a natural or biological material of interest. In some embodiments, the additive manufacturing pattern can include a printing algorithm or printing motif based on the intrinsic pattern of the mapped three dimensional spatial distribution of at least one mechanical property, material property, or structural property as well as other structural features. In some instances, the intrinsic pattern can be used to design or generate a custom-configured printing algorithm.

In other embodiments, the deposited material defines a matrix that includes plurality of pores with a hierarchal porosity and/or porosity gradient and/or porosity pattern in the composite textile. The additive manufacturing pattern for the deposited material can be based on a three dimensional spatial distribution of pores in a natural or biological material of interest.

In other embodiments, a fluid (e.g., liquid) can be provided within the pores of the composite textile. In some embodiments, the movement of the fluid in the pores can be used dissipate energy in response to force or impact on and/or of the composite textile. For example, body armor can be formed from a composite textile that includes a woven fiber substrate on which is deposited a material matrix that includes a hierarchal porosity and/or porosity gradient and/or porosity pattern. The porosity of matrix and composite textile can be such that fluid provided in the pores can dissipate impact energy or force from projectile striking the body armor.

In other embodiments, the pores can have a hierarchy and/or gradient such that composite textile includes a first region that exudes fluid in response to a compressive or tensile load and a second region that imbibes fluid in response to the load similar to the flow directing material disclosed in U.S. patent application Ser. No. 12/106,748 to Knothe Tate et al., the entirety of which is hereby incorporated by reference. The flow directing material has a porous structure and is capable of being compressed when a load is applied to the outer surfaces of the material. By way of example, the matrix defined by the deposited material can be a porous compliant polymeric material that includes a first region and the second region that extend from an outer surface of the composite textile. In response to compressive or tensile load to the composite textile, the first region can exude fluid from the outer surface toward the direction of the load, and the second region can imbibe fluid from the outer surface away from the direction of the load.

In some embodiments, the exuding region can have a first porosity, and the imbibing region can have a second porosity. The porosities (or porosity ratio (e.g., void volume of the respective region in mm³/total volume of the respective region in mm³)) of the exuding region and the imbibing region can be about 0.3 and about 0.7, respectively. The porosities of the exuding region and the imbibing region can also be at least about 5% different so that the direction of fluid flow in and/or through the exuding region will be different than (e.g., contrary, opposite, and/or substantially normal to) the direction of fluid flow in and/or through the imbibing region. That is, the difference of porosities of the exuding region and the imbibing region can determine, at least in part, the direction of fluid flow in and/or through the exuding region and the imbibing region.

The exuding region and the imbibing region can also have, respectively, a first permeability and a second permeability. The permeabilities of the exuding region and the imbibing region can be about 10⁻¹³ m² to about 10⁵ m². The permeability can control the magnitude of fluid flow in the composite textile, when the composite textile is under compression, and can potentially control the timing of transport of fluid depending on the specific application of the composite textile. In one aspect, the exuding region can have substantially the same permeability as the imbibing regions. In another aspect, the exuding region and the imbibing regions can have different permeabilities.

In other embodiments, the composite textile can include a plurality of first regions laterally spaced from one another in the composite textile and separated by the second region. At least some of the first regions can have a different porosity, volume, volumetric permeability, and/or surface permeability than the porosity, volume, volumetric permeability, and/or surface permeability of other first regions.

In other embodiments, at least one bioactive agent provided in the composite textile can include polynucleotides and/or polypeptides encoding or comprising, for example, transcription factors, differentiation factors, growth factors, and combinations thereof. The at least one bioactive agent can also include any agent capable of promoting tissue formation (e.g., bone and/or cartilage), destruction, and/or targeting a specific disease state (e.g., cancer). Examples of bioactive agents include chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., EGF), HGF, VEGF, fibroblast growth factors (e.g., bFGF), PDGF, insulin-like growth factor (e.g., IGF-I, IGF-II) and transforming growth factors (e.g., TGF-β I-III), parathyroid hormone, parathyroid hormone related peptide, bone morphogenic proteins (e.g., BMP-2, BMP-4, BMP-6, BMP-7, BMP-12, BMP-13, BMP-14), sonic hedgehog, growth differentiation factors (e.g., GDF5, GDF6, GDF8), recombinant human growth factors (e.g., MP-52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, DNA encoding for an shRNA of interest, oligonucleotides, proteoglycans, glycoproteins, and glycosaminoglycans.

The at least one cell provided in the composite textile can include any progenitor cell, such as a totipotent stem cell, a pluripotent stem cell, or a multipotent stem cell, as well as any of their lineage descendant cells, including more differentiated cells (described above). The cells can include autologous cells; however, it will be appreciated that xenogeneic, allogeneic, or syngeneic cells may also be used. Where the cells are not autologous, it may be desirable to administer immunosuppressive agents in order to minimize immunorejection. The cells employed may be primary cells, expanded cells, or cell lines, and may be dividing or non-dividing cells. Cells may be expanded ex vivo prior to introduction into the woven fiber substrate, textile, and/or fabric. For example, autologous cells can be expanded in this manner if a sufficient number of viable cells cannot be harvested from the host. Alternatively or additionally, the cells may be pieces of tissue, including tissue that has some internal structure. The cells may be primary tissue explants and preparations thereof, cell lines (including transformed cells), or host cells.

In some embodiments, the composite textile can be mixed or embedded with cells before or after implantation into the body. The composite textile can function to provide a template for the integrated growth and differentiation of the desired tissue.

In some embodiments, the cells are introduced into pores of the composite textile or textile substrate, such that they permeate into the interstitial spaces therein. For example, the composite textile or textile substrate can be soaked in a solution or suspension containing the cells, or they can be infused or injected into the matrix of the textile substrate. As would be readily apparent to one of ordinary skill in the art, the composition can include mature cells of a desired phenotype or precursors thereof, particularly to potentate the induction of the stem cells to differential appropriately within the composite (e.g., as an effect of co-culturing such cells within the composite).

In some embodiments, the composite textile can be coated on one or more surfaces, before or after consolidation with cells, with a material to improve the mechanical, tribological, or biological properties of the textile composite. Such a coating material can be resorbable or non-resorbable and can be applied by dip-coating, spray-coating, electrospinning, plasma spray coating, and/or other coating techniques. The material can be a single or multiple layers or films. The material can also comprise randomly aligned or ordered arrays of fibers. In some embodiments, the coating can comprise electrospun nanofibers. The coating material can be selected from the group including, but not limited to polypropylene, polyester, polytetrafluoroethylene (PTFE), expanded PTFE (ePTFE), polyethylene, polyurethane, polyamide, nylon, polyetheretherketone (PEEK), polysulfone, a cellulosic, fiberglass, an acrylic, tantalum, polyvinyl alcohol, carbon, ceramic, a metal, polyglycolic acid (PGA), polylactic acid (PLA), polyglycolide-lactide, polycaprolactone, polyethylene glycol) (PEG), polydioxanone, polyoxalate, a polyanhydride, a poly(phosphoester), catgut suture, collagen, silk, chitin, chitosan, hydroxyapatite, bioabsorbable calcium phosphate, hyaluronic acid, elastin, lubricin, and combinations thereof.

In some embodiments, a smooth surface coat on the composite textile is thus provided if needed. In some embodiments, the surface coat can increase durability and/or reduce friction of and/or at the surface.

In other embodiment, the textile substrate and/or composite textile can include a plurality of cuts (e.g., straight or curvilinear cuts) or folds to provide the at least one prestress and/or residual stress and/or gradient in at least one mechanical property, material property, or structural property of the composite textile. The cuts and/or fold are arranged in a kirigami pattern such that the textile substrate and/or composite textile includes at least one prestress and/or residual stress and/or gradient in at least one mechanical property, material property, or structural property of the composite textile. The kirigami-like structures may or may not be symmetrical (e.g., rotationally symmetrical).

The arrangement of cuts and/or folds of the kirigami-based textile substrate and/or composite textile may be made to a substantially two-dimensional flexible textile substrate and/or composite textile. The cuts may be straight or curved such that, when the composite textile is deflected (e.g., when the composite textile is deformed perpendicular to its original plane), a saddle-like surface develops in the regions between the cuts. The spaces between the cut ends experience substantially lower local stress and strain upon deflection compared to the spaces at the start of the cuts. Consequently, cuts and/or folds placed in different locations are subject to different mechanical deformations. The difference in deformations leads to a distribution of points on the composite textile, with some points experiencing negligible local strain that can provide the at least one prestress and/or residual stress and/or gradient in at least one mechanical property, material property, or structural property of the composite textile. This implementation is substantially improved from prior art on textiles, where intrinsic stretchability of the substrate and/or elements is commonly a limitation. Because the kirigami textile substrate and/or composite textile can originate as a flat (e.g., two dimensional) surface, a mechanoactive material formed from the textile substrate and/or composite textile, including, for example, sensor, actuators, transducers, and capacitors formed therefrom, may be more easily mass produced using robust and scalable processes. The mechanoactive material can also modular, capable of being separately processed and embedded into common garments, sports accessories, patches, etc. The stiffness as well as the prestresses and/or residual stresses of the kirigami textile substrate and/or composite textile may be tuned or otherwise selected via a number of cut parameters, as well as the selection of fiber material and thickness, casting of fibers or placement of fibers in custom loom/knitting template. The kirigami pattern can be pulled into or out of the plane, creating a third dimensional structure through which the respective warp or weft interlacing fibers can form a weave or knit.

Kirigami cutting and/or folding can also be used to engineer elasticity in the textile substrate and/or composite textile. The cutting allows for greater control over the geometric design and mechanoactive material behavior. Various technologies (e.g., laser cutting, die cutting, printing, vapor coating, etc.) may be used to generate the two-dimensional patterns, with or without functional coatings, and thereby achieve the at least one prestress and/or residual stress and/or gradient in at least one mechanical property, material property, or structural property via kirigami techniques. Curvilinear, piecewise linear, and other cut patterns may be used. The cut patterns may be useful in conforming to surfaces with complex curvature, which are useful robotic, human body (e.g., wearable), and other applications. The disclosed mechanoactive material can approximate such curved surfaces, despite deforming from a substantially planar composite textile, which may be challenging using conventional flat sheets. To address this challenge, the planar kirigami textile substrate and/or composite textiles may use repeating unit cells using fractal cuts, tessellations, cross minor, or other cuts. Lattice kirigami techniques may also be used to introduce dislocation and disclinations, which disrupt the lattice order in the textile substrate and/or composite textiles, causing out-of-plane deformation to relieve in-plain stress.

The kirigami-based nature of the mechanoactive materials may be used as a tool to geometrically manipulate the global structure and properties of materials. The kirigami textile substrates and/or composite textile may be discretized or otherwise considered as a series of beams. In this view, the segments between cuts act as hinges that cause the beams to bend out of plane with an applied stress. As a kirigami module is deformed, the beams defined by the cut lengths bend out of plane, creating a collection of saddle points with alternating positive and negative curvatures, enabling the structure to achieve large deflections. Upon cross-plane deformation, the kirigami textile substrate and/or composite textile are capable of conforming to a globally curved surface with a shape that is accommodated by the cut geometry.

In some embodiments, as illustrated in FIG. 4, the composite textile can be cut and/or folded using kirigami techniques and/or wound using gradient fibers into fingerprint textile patterns enabling sensor, actuator, transducer, capacitance/inductive functions. The warp or weft of textile substrate can be designed in classic fingerprint patterns or geometric patterns with intrinsic elasticity or stiffness; interlacing, weaving, knitting, knotting to cross link in the respective weft or warp directions.

The pattern can be formed via kirigami or casting of fibers or placement of fibers in custom loom/knitting template. The kirigami pattern can be pulled into or out of the plane, creating a third dimensional structure through which the respective warp or weft interlacing fibers can form a weave or knit.

In some embodiments, the kiragami element of the composite textile can serve, similar to an atomic force microscopy probe tip, as a sensor/actuator/transducer, where mechanical properties at the interface can be spatially probed. The spiral (either round or square or other shape) and zig zag or other shape of the kiragami is in this way ‘suspended’ in the weave and its deformation, e.g., during wear of the textile, can be decoded to give spatial maps of interface surface displacements and/or force distributions at surfaces and/or strains at surfaces. If the element itself has topography and shape and mechanical properties emulating that of fingertips (friction ridge patterns with loops, whorls and arches), the textile can be used to emulate and replicate tactile sensory patterns for haptic applications.

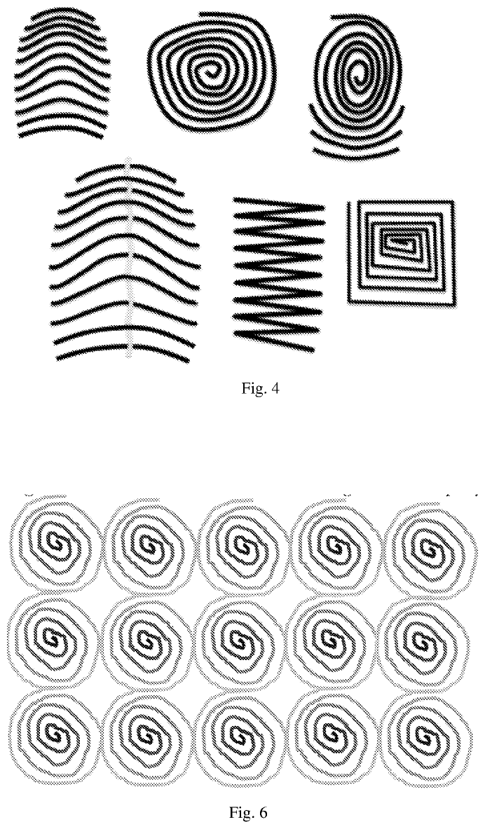

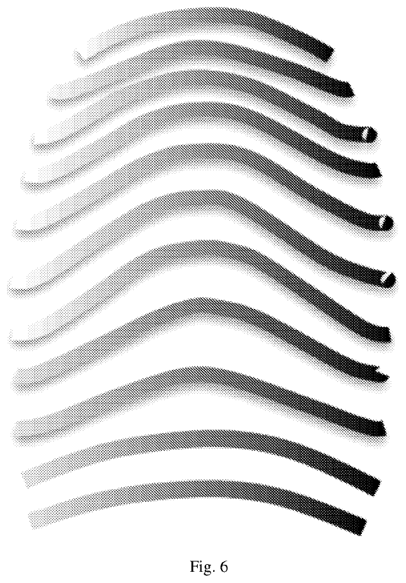

In another embodiment, as illustrated in FIG. 5, composite textiles having fingerprint patterns can be grouped spatially to achieve specific mechanoactive effects. The fibers within the patterns can be tuned to exhibit specific stiffnesses and/or elasticities along each individual fiber, to achieve regional mechanoactive elements and or gradients in properties.

It will be appreciated the composite textile can be used in the formation a variety of mechanoactive materials where it is desired to control or modulate mechanical properties of the material. Such mechanoactive materials can include body armor, tissue constructs, and wound dressings as described herein as well as other materials, such as flooring material, where it is desirable to provide strength in tension and bending with smart poroelastic properties, found in flow directing materials. Additionally, mechanoactive materials including the composite textiles can be used to form wearables, such as clothing, garments, or dressings, that can dynamically apply pressure in various points of the body to increase or decrease blood flow, imbibe or exude moisture, based on external stimuli.

In some embodiments, the composite textile so formed can be used to generate engineered tissue implant or mechanically functional textiles, which can be used to treat and/or repair tissue defects, such as bone defects or soft tissue defects. The composite textile can be used in its native form in combination with other materials, as an acellular (non-viable) matrix, or combined with at least one cell and/or at least one bioactive agents (e.g., growth factors) for use in repair, regeneration, and/or replacement of diseased or traumatized tissue and/or tissue engineering applications. An advantage of the presently disclosed subject matter is the ability to produce biomaterial scaffolds and composite matrices that have precisely defined mechanical properties that can be inhomogeneous (vary with site), anisotropic (vary with direction), nonlinear (vary with strain), and/or viscoelastic (vary with time or rate of loading) and that mimic native or natural tissue to be treated and/or repaired.

In some embodiments, the composite textile can be employed in any suitable manner to facilitate the growth and generation of desired tissue types or structures. For example, the composite textile can be constructed using three-dimensional or stereotactic modeling techniques. Thus, for example, a layer or domain within the composite textile can be populated by cells primed for one type of cellular differentiation, and another layer or domain within the composite textile can be populated with cells primed for a different type of cellular differentiation. As disclosed herein and as would be readily apparent to one of skill in the art, to direct the growth and differentiation of the desired structure, in some embodiments, the composite textile can be cultured ex vivo in a bioreactor or incubator, as appropriate. In some embodiments, the structure is implanted within the subject directly at the site in which it is desired to grow the tissue or structure. In further embodiments, the composite textile can be grafted on a host (e.g., an animal such as a pig, baboon, etc.), where it can be grown and matured until ready for use, wherein the mature structure is excised from the host and implanted into the subject.

It will be appreciated that the composite textile can be used in a variety of engineered smart materials bespoke external (wearable) and internal (implants, medical devices) wound dressings that deliver drugs and take up wound exudate.

In some embodiments, a wound dressing can be formed from a composite textile that includes a textile substrate and a porous matrix. The textile substrate can have a region of temporally-controlled elasticity that transitions between a first state and a second state in response to the external stimuli. The first state can be more relaxed than the second state, and the smart material can at least partially revert from the second state to the first state over an extended time period resulting from the temporally-controlled elasticity of the textile substrate. The internal energy of the smart material in the first state can be less than internal energy of the substrate in the second state. Different regions of the smart material can possess different temporally-controlled elasticity.

The porous matrix of the composite textile can both imbibe excess fluid or exudate from a wound and exudes therapeutic agents to the wound when the dressing is under compression. In some embodiments, the substrate includes a plurality of laterally spaced exuding regions in the form of cylindrical dots that under compression exude a therapeutic fluid. The material surrounding the dots can imbibe excess fluid or exudate when the dressing is compressed against the wound. The exuding regions have a first porosity and a first permeability. The imbibing surrounding region has a second porosity and a second permeability.

The exuding regions of the dressing can include depots (not shown) that contain the therapeutic fluid in the exuding regions. The therapeutic fluid can flow from the exuding regions through a delivery surface when the dressing is under compression. The therapeutic fluid can include at least one pharmaceutical agent, anti-inflammatory agent, antibiotic, antifungal agent, antipathogenic agent, antiseptic agent, hemostatic agents, local analgesics, immunosuppressive agents, growth factor, peptide, or gene therapy agent. The second imbibing region can imbibe excess fluid or exudate from the wound or skin of the subject when the delivery surface of the dressing is applied against the wound or skin of the subject and compressed.

The exuding regions of the dressing can comprise a first porous polymeric material having a first porosity. The surrounding imbibing region can comprise a second porous polymeric material having a second porosity different that the first porosity. The first porous polymeric material can have a first flexible polymeric foam structure of interconnected open cells. The second porous polymeric material can have a second flexible polymeric foam structure of interconnected open cells.

The dressing can also include a slip layer attached to the outer surface of the substrate. The slip layer can minimize friction of the dressing with the outer environment when the dressing is applied to a wound of the subject.

The composite dressing can deliver therapeutic substances through the delivery dots and imbibe fluid through the surrounding material surrounding the dots. The composite dressing can also be designed and/or deliver substances through the larger volume material surrounding the dots and imbibe fluid through the smaller volume of the dots.

### EXAMPLE

Nature abounds with stimuli-responsive, so-called smart materials. Examples of such materials, at the macro- to meso-length scale, include skin, bones, and skin-on-bones (periosteum) in the animal kingdom, and eucalyptus tree bark, cambium, and wood in the plant kingdom. Connective tissues comprising skin and other soft animal tissues exhibit remarkable mechanical strength, functional barrier properties to prevent moisture loss to the environment, while also “waterproofing” the internal organs, as well as self-healing and -sensing (e.g., pressure sensing) capacities. Vascular tissues of trees generate hydraulic pressure pulses when they bend in the wind and bone exhibits flow directing properties under mechanical loading, emerging from different calibers of interconnected vascular, pericellular, and matrix porosities. While top-down approaches to designing and manufacturing such smart materials have met with little success, bottom-up approaches using paradigms of “cellular manufacture” have been met with great success.

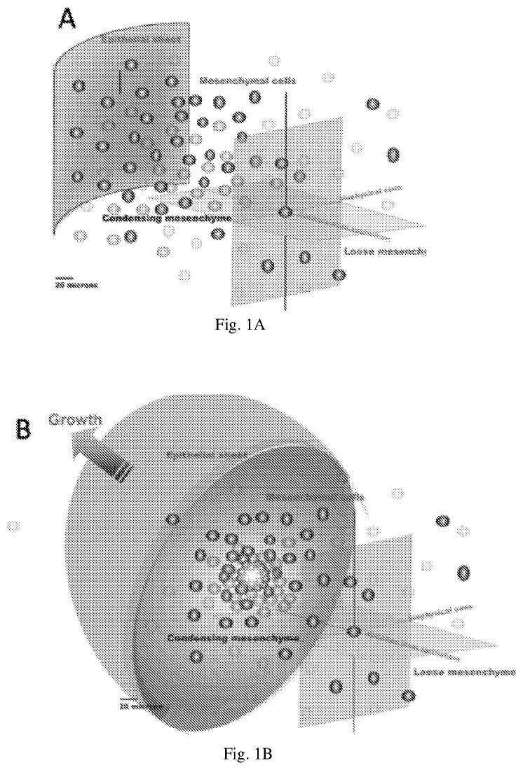

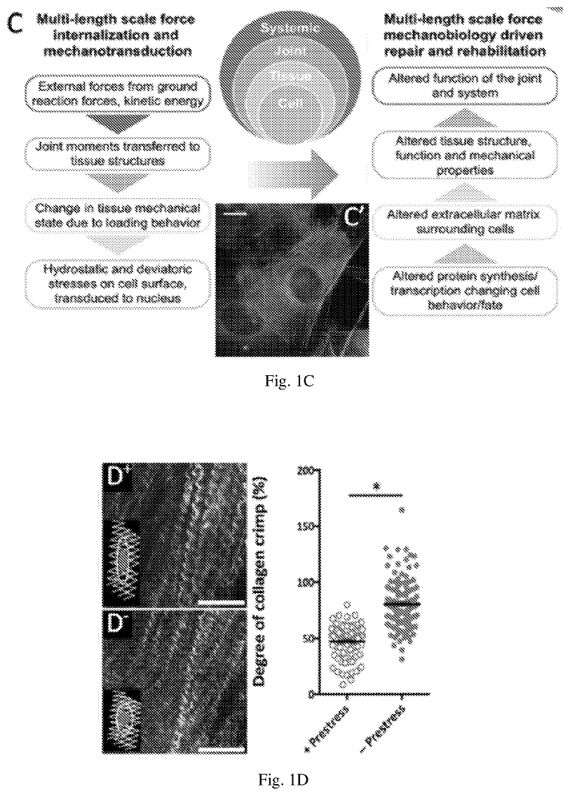

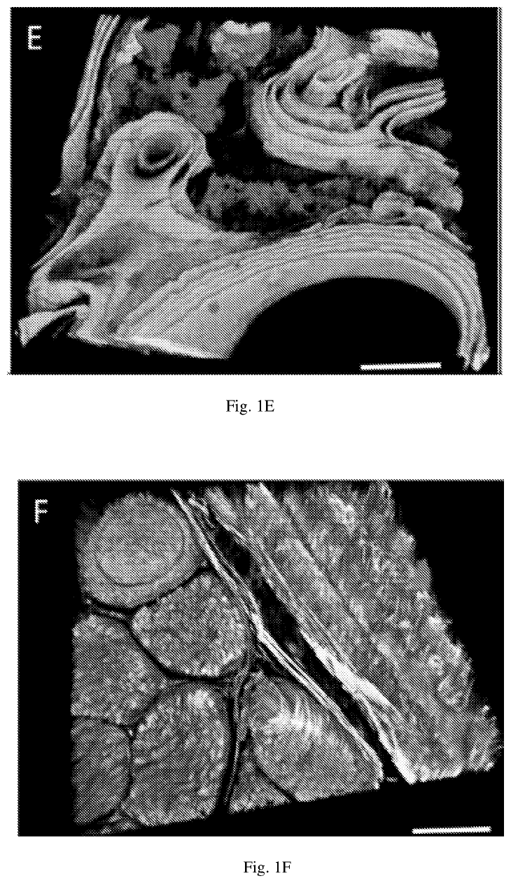

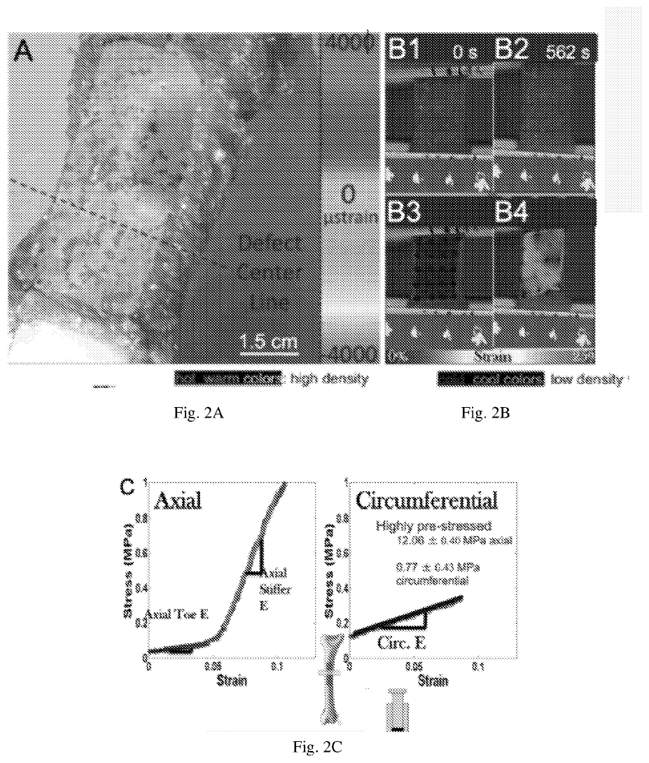

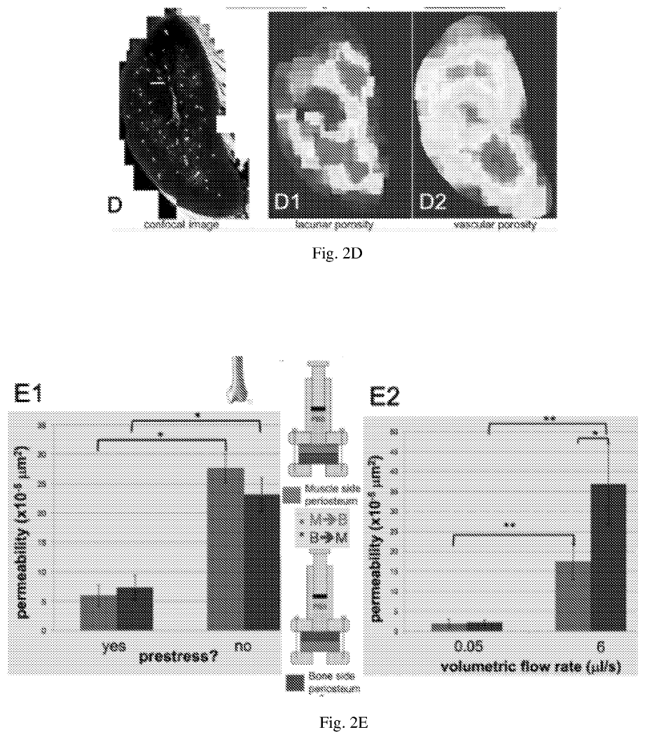

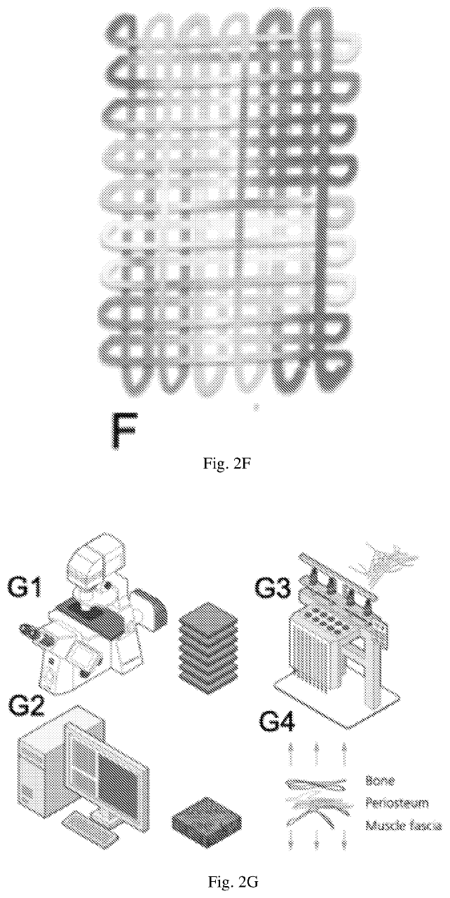

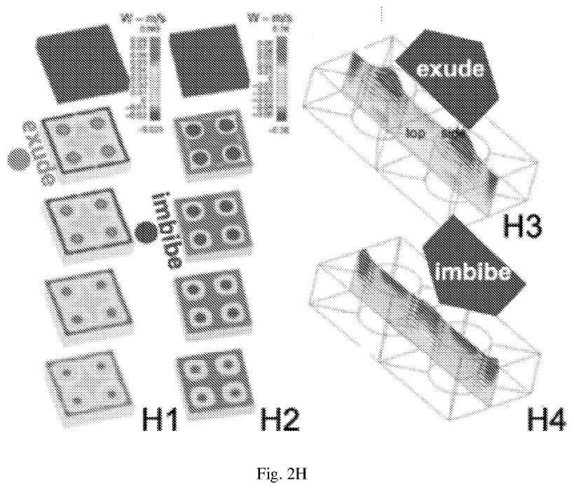

Remarkably, the “brainless” cells that manufacture all of the aforementioned smart materials, themselves form living sensors, actuators and transducers at the nano- to micron length scale. The advent of imaging across length- and time-scales has enabled not only unprecedented elucidation of the mechanisms underpinning cells' and natural materials' smart properties (FIG. 1), but also the design and manufacture of new materials emulating nature's own (FIG. 2). MADAME refers to a computer-aided additive manufacturing platform that incorporates Multi-D printing and/or computer-controlled weaving to create novel, bio-inspired materials and products. The state of the art imaging capacity enables observation of live cells in their native tissue habitats. Design thinking processes empower engineers to empathize with their cells, imagining and feeling what they experience and envisioning their responses. In empathizing with their cells, engineers may be better equipped to prototype mechanoactive materials and architectures as cells do, from raw materials that they themselves produce and adapting their own structure and function, and ultimately their own niche to survive. This perspective expands upon and integrates these topics to establish a foundation for advanced design and manufacture of mechanoactive materials that will be relevant for fields of use as varied as the medical and transport sectors, as well as for external and internal applications.

### Cells as Sensors, Actuators, and Transducers

Throughout nature, cells are the master designers, manufacturers and builders of tissue architectures underpinning e.g., trees and their population forests in the plant kingdom, as well as organs and organismal systems in the animal world. Cells literally differentiate themselves in their degree of differentiation, a biological term for structural and functional specialization. From undifferentiated stem cells, to terminally differentiated cells as diverse as dendritic bone and brain cells+, the structure and architecture of the cell encodes structural and functional memories of the cell's life experience. Repeated activities reinforce structural connections (e.g., of the cytoskeleton, cell-matrix and cell-cell connections, etc.) which are stabilized over time, resulting in an adaptive functional memory of cell's experiences throughout life. Cells' dynamic stability and agility are a function of the cells' structural stability as well as adaptive capacity, in context of cellular time scales for e.g., division, motility, structural protein expression and secretion, etc. The cytoskeleton is one repository of cellular information and the totality of cell structures at any given moment in time serve as an integrated sensor for the health state of the tissue and/or organ at that point in time and geographic location. Since cells sense their local environment and transduce biophysical (mechanical, electrical, osmotic, etc.) information to the nucleus, where gene up- and down regulation leads to stabilization of the cell and/or the cell's environment over time, the cell itself is also an actuator of structural and architectural change as well as new local and global equilibria (FIG. 1C). By sensing and transducing information from its environment to the nucleus, where the basic building block proteins of tissues are created and secreted vectorially (with a magnitude/concentration and direction) to the extracellular matrix, the cell actively influences force balances at all relevant interfaces—between cells, cells and the matrix, and even between the cytoplasm and the nucleus. Cells are indeed smart in a materials science context, if not in a brain science context.

Tissues Cells Weave and their Smart Properties

Natural materials such as animal and plant tissues are natural composites comprising resilient collagens (animals) and celluloses (plants) that confer toughness, elastin (animals) and elastin-like polypeptides (plants) that impart elasticity, and proteoglycans that bind water and give turgidity. Cells “spin and weave” components of tissues in situ—the remarkable stimuli-responsive (smart) and adaptive properties of tissues emerge macroscopically from the directional, cellular secretion of nanoscopic extracellular matrix proteins as well as their anisotropic multicellular assembly and dis-assembly (polymerization and depolymerization). While in the future, the capacity to guide cells to manufacture smart materials in a controlled way may become possible, the current focus of this author's R&D program is on emulating nature's paradigms, either through scaled-up solutions based on similitude theory or recursive logic based approaches. An overview is provided below, with specific examples inspired by skin, bone, and skin-on-bones in the following sections.

### Geometry

The success of Lego building blocks, with their universal interlocking mechanism, is their flexibility to build structures only limited by the creative capacity of the human brain. Nature's counterpart could be the cells' capacity to organize into sheets (epithelial sheet, FIG. 1A) and three-dimensional, mesenchymal-epithelial transition (MET).

Tissues deriving developmentally from the epithelial sheets provide essential barrier functions to tissues at interfaces between different environments, e.g., skin provides a barrier between richly hydrated tissues of the body (85% water by volume) and the outside environment and the skin-on-bones periosteum covers all non-collagenous bone surfaces of the body, separating the interior milieu from the outside environment of bone, e.g., muscle compartments, fat, etc. (FIGS. 1F, 2A, B). Tight junctions between cells of epithelial sheets underpin this barrier property with their interpenetrating, zipper-like closures between cell membranes.

In contrast, more globular mesenchyme consists of cells and extracellular matrix, joined by a variety of cell-cell and cell matrix junctions that act as molecular rivets and more distributed Velcro-like attachments that each, respectively, attach to proteins of the cell's own skeleton (the cytoskeleton). Mesenchymal condensation, an event that occurs 11.5 days after fertilization in the mouse, initiates the formation of the musculoskeletal system. During this event, cells and their nascent tissues begin to specialize and rapidly scale-up cell number through cell division (proliferation) and formation of tissue templates through production of extracellular matrix. Throughout the process, new geometries and architectures are stabilized by cell-cell and cell-matrix junctions.

Throughout prenatal development as well as during postnatal healing, which repeats prenatal development processes, cells interconvert between epithelial and mesenchymal states (EMTs and EMTs, as per above), enabling the formation of complex geometries, tissue architectures, and organ systems (FIGS. 1A, B). The process is so ubiquitous that the formation of deleterious tissues such as cancerous tumors and metastasis of circulating tumor cells follow analogous paradigms. In many ways, this interconversion of geometries provides a most basic archetype for tissue architectures.

### Mechanical Modulation of Polarity to Anisotropy

Just as mechanical forces modulate EMTs and METs as tissue architectures evolve, they also direct self assembly of cell layers at the earliest stages of tissue organization. Forces including cell adhesion and cell tension modulate tissue patterning of tissues and influence tissue phenotype from the time of fertilization until after birth and throughout life. As mechanisms by which forces inherent to life on Earth translate to self-assembly of multicellular structures, including tissues and organs, this knowledge provides direct inspiration for bottom-up design, engineering and manufacturing of mechanoactive materials and devices (FIGS. 1C, 2F, G). Also of great relevance, from the earliest stages of cell polarization to formation of materials exhibiting anisotropy, mechanical interactions and concentration gradients, at the interface of the cell and its environment, guide the transcription and secretion of structural proteins, the building blocks of tissues, in anisotropic structures that translate to anisotropic (in terms of mechanical properties) or directional functions and properties.

### Mechanoactive Materials

Electroactive polymers and materials change shape or size under electrical stimulation, while mechanoactive fibers and materials exhibit stimuli-responsive (smart) properties under mechanical loading (FIG. 2). Given that life itself is mechanobiological, it is surprising that mechanoactive materials are less recognized and researched than electroactive materials. For example, if one compares search engine results for mechanoactive and electroactive materials, in PubMed, the former garner 14 compared to 4000 hits and, via google, 11,000 versus 2,200,000 respective results. The plethora of electroactive materials and applications parallels industry and manufacturing activities around the development of electronics and electronic components, and their scale down in size over time. In the future, one would expect increased publication and patent activity around scaling down of advanced materials' and device manufacturing to include smart properties that emerge from smaller to larger length scales.

Interestingly, while top-down engineering approaches have led to elucidation of multiscale structure-function relationships in a variety of natural materials and tissue types, bottom-up approaches appear more conducive to the elucidation, engineering and manufacture of emergent mechanoactive properties. Emergence refers to properties or patterns that arise from the putting together of simpler elements which in themselves do not exhibit similar properties or patterns. The concept is further explained by example below.

### Periosteum, the Skin-on-Bones

Periosteum, a hyper-elastic soft tissue sleeve, envelops all non-particular (excluding the joint surfaces covered in cartilage) bony surfaces of the body, like a “skin-on-bones.” As an inherently “smart” material, soft periosteum imparts hard bones with added failure strength under high impact loads. Recent studies of periosteum's mechanoactive properties reveal cellular and structural mechanisms underpinning its myriad smart properties, including anisotropic stiffness (modulus of elasticity), direction-dependent intrinsic prestress and permeability, and a stem cell-triggering molecular weave that switches state upon release of prestress.

First, high definition television (HDTV) lenses were used to map in high resolution (submicron scale), four-dimensional (xyzt) strains in periosteum during stance shift loading of the sheep femur. The rationale was to understand the local environment of stem cells that reside in a quiescent state in the periosteum until injury occurs. The working hypothesis was that the cells sense local strains in the periosteum which trigger them to egress from the periosteum in injury-inducing loading scenarios. Interestingly, 4D-imaging of strains in the periosteum revealed surprising heterogeneity in space and time (FIG. 2A). Furthermore, areas of new tissue genesis via stem cells egressing from the periosteum correlate to areas with the largest shift in baseline strain rather than absolute strain magnitudes.

Further paired imaging-mechanics studies on sections of periosteum from the longitudinal (axial, along the long bone) and/or circumferential, anterior (front) aspect of the sheep femur demonstrated a stark anisotropy in mechanical properties of the periosteum. When loaded in tension, axial oriented sections (FIGS. 2B, C-Axial), exhibit significant strain stiffening above 0.05, while circumferentially oriented sections (FIGS. 2B, C-Circumferential) exhibit linear elastic behavior.

Periosteum is attached to all bony surfaces via a plethora of collagen connections called Sharpey's fibers, which “Velcro” the soft tissue to the hard bony surface. When injured, or during orthopedic surgeries involving periosteal lifting, the Sharpey's fibers become severed. In parallel with the mechanics studies described above, shrinkage of periosteum upon release from the underlying bone surface was quantified. Similar to mechanical stiffness, tissue shrinkage exhibited direction-dependence, whereby upon release, the tissue sections shrank significantly more in the axial compared to the circumferential direction. These relative shrinkages were used to calculate an intrinsic prestress in the tissue (FIG. 2C), revealing that the tissue is highly prestressed in the axial direction (12.06 0.40 MPa) and much less so in the circumferential direction (0.77 0.43 MPa).

Follow on high resolution microscope imaging studies revealed a potential mechanical trigger for quiescent stem cells to activate healing processes. By probing the interaction of light with the molecular structure of periosteum [second harmonic and two photon imaging, submicron resolution maps of collagen and elastin fibers in the tissue were rendered. When applied to freshly excised bone and periosteum in situ, it was observed that the intrinsic crimp or curl of collagen fibrils relaxes when the periosteum is released from the underlying bone via cutting of the Sharpey's fibers (FIG. 1D). The relaxation in the curl was observed to coincide with rounding up of the resident stem cells adhering to the tissue fibers, providing a direct trigger for the cells to revert from quiescent to active states, and to initiate the genesis of new tissue templates associated with postnatal healing and prenatal development.

Excitingly, the release of the tissue's intrinsic prestress had a significant impact on another property of the tissue, namely its permeability. Permeability is an essential functional boundary property (FIG. 2E1) for periosteum, which serves as an interface between bone and surrounding muscle. Release of prestress in the tissue was associated with a significant reduction in permeability of periosteum. Furthermore, permeability of tissue-similar fluid (Ringer's lactate) through the periosteum exhibited direction- and flow-rate dependence. Permeability of periosteum increased eight to 16 when the flow rate was increased 120. Surprisingly, this effect was much more pronounced in the bone to muscle direction than in the muscle- to-bone direction, a characteristic of a non-linear hydraulic valve.

### Bone

Further smart properties of bone reveal themselves when one analyzes the different phases of the tissue itself. During development in utero and during postnatal healing, bone starts as a soft template of collagen and elastin that mineralizes over time. The temporal aspect of bone mineralization and maturation can be tracked using fluorochromes of different excitation and emission wavelengths (FIG. 1E; different spectra excite different fluorophores). The fluorochromes chelate chemically to the mineral when it is laid down onto (nucleates around the fibrils of) the bone template; intramuscular or subcutaneous injection of fluorochromes over the 4-month-healing cycle reveals the temporal dynamics of mineralization, e.g., of a tissue template formed via periosteum-derived stem cells ingressing into a bone defect (FIG. 1E). Whereas initial mineralization forms a disorganized scaffold referred to as woven bone (FIG. 1E), subsequent tissue genesis and mineralization occurs layer by layer, typically in proximity to the vascular supply (green and then turquoise, in time intervals comprising weeks, FIG. 1E).

Intrigued by the observation of counterintuitive flows in experimentally based computational models of bones, we aimed to test the hypothesis that non-homogeneous distributions of different caliber pores in bone would result in such counterintuitive flows, e.g., imbibement of fluid under compression. Using high resolution microscopy, bone's different caliber porosities (pericellular versus vascular pores) were rendered as heat maps with warm colors depicting areas of high density and cool colors showing areas of low density (FIG. 2D. We then re-ran our computational models, which showed that specific patterns of porosity of different calibers indeed confer emergent, flow-directing properties to the tissue under mechanical loads.

### Microscopy-Aided Design and Manufacture (Madame) of Bio-Inspired Materials and Structures Concept—Recursive Logic to Emulate Natural Tissues

Once we were able to quantify and precisely describe, quantitatively in four dimensions (4D), multiscale structure and emergent functional properties of natural materials, we then aimed to develop methods to design and manufacture new materials emulating nature's own. This resulted in the development of a novel process, referred to as MADAME, to map spatial and temporal properties of smart, natural materials (FIGS. 2F, G). The process uses imaging and advanced computational methods to visualize patterns intrinsic to the material (FIGS. 1, 2). Using recursive logic, the basis of computer algorithms, these patterns are recreated digitally using computer-aided design principles, and physically using computer-assisted weaving and/or knitting, alone and/or in combination with multidimensional additive manufacturing, e.g., 3D printing, stereolithography and laser sintering.

The Jacquard loom was the earliest computer—in 1801, a century prior to “the first punch card driven computers, the Jacquard loom wove patterns using loops of paper with holes to guide when hooks fell through the paper loop (hook down) or stayed above the loop (hook up), thereby encoding binary patterns of e.g., tapestry weaves”. The computer-controlled Jacquard looms and additive manufacturing systems enable creation of physical embodiments (textiles, composites) of mechanical and other biophysical and spatiotemporal patterns intrinsically encoded in natural materials.

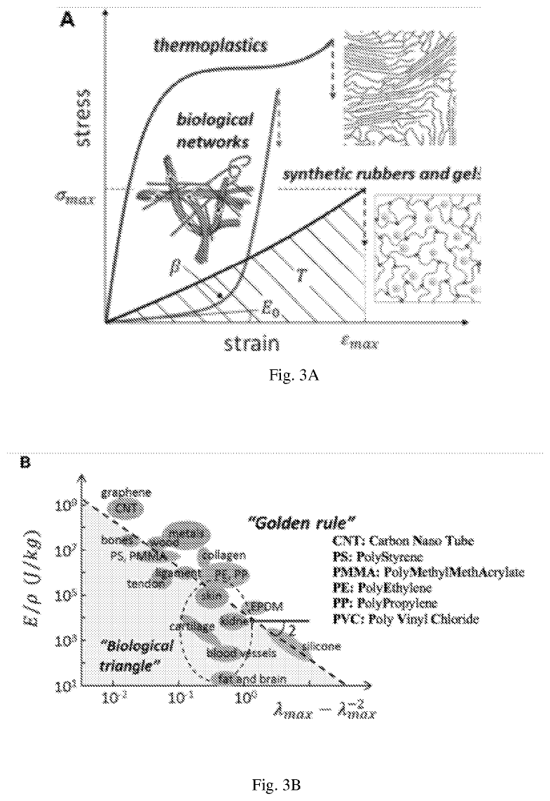

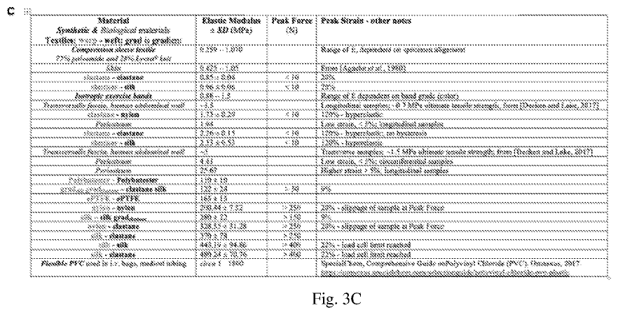

More recent approaches suggest application of recursive logic for polymer design and engineering to mimic a range of mechanical properties appropriate for biological applications (FIG. 3). While reduction to practice is currently in homogenous materials without intrinsic anisotropy or mechanical gradient properties, the concept expands upon the idea of encoding material properties architecturally, at molecular length scales, to achieve a range of biologically relevant mechanical properties previously achievable only empirically, through mixing of “various polymers, solvents and fillers”. In combination with the aforementioned MADAME approach, a range of architectures might be achievable that range from molecular to micro- to macro- and meso-length scales. In particular, molecular encoding of polymers may provide a means to tune fiber and matrix mechanical properties for composite, advanced manufactured materials and products. In the future, it may enable real time manufacture of gradients materials through adjustment of the molecular composition of e.g., 3D printers and other advanced manufacturing platform materials.

### Incorporation of Pre- and Residual Stresses in Mechanoactive Material Design and Manufacture

Once we implemented additive manufacturing with textile engineering to create composite structures, we aimed to integrate prestresses and/or residual stresses, found in natural materials, into advance-manufactured smart materials. A two-pronged solution proved most effective. First we used a pre-tensioning system with the Jacquard loom to selectively prestress warp and weft (orthogonal weave) fiber directions. Then we incorporated principles of Kirigami, the Japanese art of folding and cutting paper into shapes, to cut and/or preform fibers and/or fiber composites with defined residual stresses and dimensions, from sheets or composites of desired materials. Stresses were thereby defined by the geometry of the cuts, e.g., spiral versus zig-zag with characteristic dimensions defining magnitudes of stresses and their gradients, or by the architecture of the composite fibers. While kirigami has been applied in electroactive materials, its use in mechanoactive materials is novel, in particular in combination with weaving, knitting and additive manufacturing applications. In developing these mechanisms and manufacturing strategies (below), the design thinking approach of empathizing with one's cells is particularly useful to brainstorm for new design elements and methods of reducing them to practice.

### Manufacturing Considerations

Application of design thinking approaches that use the visualization tool of empathizing with cells that manufacture tissues, stimulates conceptualization of new manufacturing processes and/or pipelines. When one observes a range of natural architectures across phyla of the animal and plant kingdoms, from bone to corals (animal kingdom), and wide-ranging plants, the aforementioned geometric and/or directional/anisotropic paradigms are reiterated again and again, and thereby create an infinite number of structures and associated mechanoactive functions. In summary, empathizing with cells and understanding their unique structural and functional capacities facilitates development of new manufacturing methods and processes at different length and time scales. While surface based growth of biological structures is rapidly emulatable in additive manufacturing, new manufacturing modalities need to be developed further for other architectures and anisotropic properties. These manufacturing pipelines may incorporate steps typically used in composite structure and sandwich structure design, with modular assembly or they may involve novel incorporation of mechanoactive fiber assembly into textiles which themselves are assembled within photopolymerizable resin matrix and/or sintering powders. In other words, multimodal advanced manufacturing will likely follow in the footsteps of multimodal imaging, according to the MADAME paradigm described above. In materials not incorporating living cells, clever designs can be invented to actively modulate force balances at interfaces, much in the ways that cells do in living materials.

Mechanoactive materials first found applications in tissue engineering for the development of tissue templates and scaffolds, as a direct translation from its inspiration. In parallel, the mechanoactive materials were introduced for internal (implants) and external medical devices, mainly in cardiovascular and orthopedic fields of use. New classes of mechanoactive materials will find uses in the medical and health sectors, in addition to safety and transport, military, sports, and leisure wear sectors, cementing their disruptive status in the field of material science and advanced manufacturing.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1-26. (canceled)

27. A method of forming a mechanoactive material, the method comprising:
assembling a plurality of fibers based on a fiber assembly pattern into a textile substrate; and
depositing a material via an additive manufacturing technique onto and/or between fibers of the textile substrate based on an additive manufacturing pattern to provide a composite textile that includes at least one prestress and/or residual stress and/or exhibits a change in at least one mechanical property, material property, or structure in response to at least one endogenous and/or exogenous stimulus.

28. The method of claim 27, wherein the prestresses and/or residual stresses in the composite textile provide spatially and/or temporally resolved regions to sense, apply, transduce, release, and/or store at least one of forces, displacement and/or energy when triggered and/or activated.

29. The method of claim 27, wherein the fiber assembly pattern includes different biophysically responsive fibers in sections of the textile substrate, the different biophysically responsive fibers providing at least one prestress and/or residual stress in the composite textile.

30. The method of claim 27, wherein composite textile includes areas of prestresses and/or residual stress and/or bulk prestresses and/or residual stresses.

31. The method of claim 27, wherein the at least one prestress and/or residual stress in the composite textile is defined by at least one of the shape, material, and/or stiffness of the fibers.

32. The method of claim 27, wherein the fiber assembly pattern includes selectively prestressed warp and weft fibers.

33. The method of claim 27, further comprising cutting and/or folding the composite textile to generate two dimension and/or three dimensional cut patterns that provide at least one prestress and/or residual stress in the composite textile and/or gradient in at least one mechanical property, material property, or structural property of the composite textile.

34. The method of claim 27, further comprising mapping a three dimensional spatial distribution of at least one mechanical property, material property, or structure of a natural or biological material of interest; and
designing the fiber assembly pattern and/or the additive manufacturing pattern based the intrinsic pattern of at least one mechanical property, material property, or structural property of the biological material of interest.

35. The method of claim 27, wherein the fiber assembly pattern is based on fingerprint patterns or geometric patterns with intrinsic elasticity and/or spatially distinct topography.

36. The method of claim 27, wherein the fibers include a warp and weft that are interlaced, woven, knitted, and/or knotted into the fiber assembly pattern.

37. The method of claim 27, wherein the assembled fibers are woven using a weaving algorithm based on the intrinsic pattern to define the weave pattern and fiber orientation.

38. The method of claim 27, wherein additive manufacturing technique comprises one or more of a fused deposition modeling (FDM) technique, a fused filament fabrication (FFF) technique, a big area additive manufacturing (BAAM) technique, a robocasting technique, a paste extrusion technique, casting technique, and/or a direct ink writing (DIW) technique.

39. The method of claim 27, wherein the deposited material defines a matrix that includes plurality of pores with a hierarchal porosity and/or porosity gradient in the composite textile.

40. The method of claim 27, wherein the additive manufacturing pattern is based on a three dimensional spatial distribution of pores in biological material of interest.

41. The method of claim 39, further comprising providing a fluid within the pores, the movement of the fluid in the pores dissipating energy in response to force or impact of the composite textile.

42. The method of claim 27, the composite textile including a plurality of first regions spaced from one another in the composite textile and separated by second regions wherein the first regions and second regions differing in at least one of a mechanical property, material property, or structural property.

43. The method of claim 40, wherein at least some of the first regions having a different porosity, volume, volumetric permeability, and/or surface permeability than the porosity, volume, volumetric permeability, and/or surface permeability of other first regions.

44. The method of claim 27, wherein the composite textile has a region of temporally-controlled elasticity that transitions between a first state and a second state in response to the external stimuli, wherein the first state is more relaxed than the second state, and the composite textile can at least partially revert from the second state to the first state over an extended time period resulting from the temporally-controlled elasticity of the textile substrate.

45. The method of claim 44, wherein internal energy of the composite textile in the first state is less than internal energy of the composite in the second state.

46. The method of claim 44, wherein different regions of the composite textile possess different temporally-controlled elasticity.

47. The method of claim 44, wherein the composite textile moves from the second state to the first state via any one of elongation or shortening of the composite textile, or relaxation or stiffening of the composite textile.

48. The method of claim 44, wherein the textile substrate possesses spatially-controlled elasticity, whereby different regions of the composite textile have different elasticity.

49. The method of claim 44, wherein the textile substrate is woven using at least two threads/fibers, wherein each thread has a different elasticity.

50. The method of claim 44, wherein the textile substrate includes at least one thread possessing elasticity that varies along the length of the thread.

51. The method of claim 44, wherein the textile substrate includes at least one thread possessing elasticity that varies within the cross-section of the thread.

52. The method of claim 44, wherein the textile substrate is woven using threads arranged in different directions such that the threads move frictionally relative to one another causing the transition from the first state to the second state to occur over an extended time period.
